(19) United States
(12) Patent Application Publication
(10) Pub. No.: US 2026/0297636 A1
(43) Pub. Date: Oct. 1, 2026

(54) METHODS OF PRODUCING JASMONATES

(71) Applicant: Agency for Science, Technology and Research, Singapore (SG)

(72) Inventors: Martin Patrick PAGAC, Singapore (SG); Antony Sagayaraj IRUDAYASWAMY, Singapore (SG); Thomas Larry DAWSON, Jr., Singapore (SG)

(73) Assignee: Agency for Science, Technology and Research, Singapore (SG)

(21) Appl. No.: 18/996,363

(22) PCT Filed: Jun. 23, 2023
(86) PCT No.: PCT/SG2023/050446

§ 371 Date: Jan. 17, 2025

(30) Foreign Application Priority Data
Aug. 11, 2022 (SG) 10202250708N

(51) Int. Cl. C12P 7/62; C12N 1/16; C12N 9/10; C12N 9/22; C12N 15/11; C12R 1/645

(52) U.S. Cl. CPC C12P 7/62; C12N 1/16; C12N 9/1029; C12N 9/226; C12N 15/111; C12N 2310/20; C12R 2001/645

(57) ABSTRACT

The invention relates to a method of producing a Jasmonate using a fungal cell wherein the expression of the polyketide synthase (PKS) gene has been disrupted in the fungal cell. In one embodiment, the fungal cell is yeast cell Malassezia species.

### FIELD OF INVENTION

The invention relates generally to the field of microbial biotechnology. In particular, the invention relates to methods for producing Jasmonates in a fungal cell.

### BACKGROUND

Biocontrol agents (BCA) are living organisms and/or derivatives that protect plants against diseases and pests by direct anti-pathogenic effects or indirectly via induction of host resistance. Jasmonic acid (JA) and methyl-Jasmonate (MeJA) are plant hormones belonging to a large phyto-oxylipin family collectively termed Jasmonates. Jasmonates are derived from enzymatic oxygenation of polyunsaturated fatty acids (PUFA), exhibit direct biocidal activities, and are involved in diverse signaling processes including regulation of plant defence mechanisms against insect and pathogen attack and stress reduction. In addition, JA or MeJA application to crops induces a plethora of beneficial effects including infection resistance, increased production of antioxidant activity, growth improvement, seed germination, enhanced stress and drought tolerance, and photosynthetic and transpiration rate. Jasmonates are potent, with a picomolar effective range sufficient to induce downstream effects. Jasmonates are also important flavor and fragrance compounds for multiple food and consumer care products.

There is increasing worldwide environmental safety and sustainability awareness, including concerns about excessive utilization of pesticides and fertilizers in agricultural practice. In order to reduce the impact of pesticide and fertilizer overuse on our environment, the alternative use of BCAs such as JAs to induce crop resistance against pathogens and insects has drawn significant investment by many companies. Unfortunately, Jasmonate production costs can be exorbitant, leading to jasmonate prices which run into the thousands per gram, and this limits widespread adoption of Jasmonates as alternative agrochemicals. Moreover, to yield 1 kg of essential oil containing approximately 1 g of MeJA, 500 kg of Jasminum grandiflorum petals, corresponding to 10,000 blossoms, are required. Not only does this time-consuming and labour extensive process drive the high price, but the production methodology is also highly unsustainable.

Accordingly, it is generally desirable to overcome or ameliorate one or more of the above-mentioned difficulties.

### SUMMARY

Disclosed herein is a method of producing a Jasmonate using a fungal cell, the method comprising a) culturing a fungal cell under conditions suitable for producing the Jasmonate, wherein expression of the polyketide synthase (PKS) gene has been disrupted in the fungal cell, and b) isolating a Jasmonate produced from the fungal cell.

Disclosed herein is a method of enhancing the production of a Jasmonate from a fungal cell, the method comprising disrupting expression of the polyketide synthase (PKS) gene in the fungal cell.

Disclosed herein is an engineered fungal cell for producing a Jasmonate, wherein the polyketide synthase (PKS) gene has been disrupted in the fungal cell.

Disclosed herein is a microbiome composition comprising a fungal cell, wherein the polyketide synthase (PKS) gene has been disrupted in the fungal cell.

Disclosed herein is a microbiome composition as defined herein for use as a cosmetic or as a medicament.

Disclosed herein is the use of a microbiome composition as defined herein for producing a Jasmonate.

### BRIEF DESCRIPTION OF DRAWINGS

Embodiments of the present invention are hereafter described, by way of non-limiting example only, with reference to the accompanying drawings in which:

FIG. 1 shows an LC-MS/MS chromatogram of jasmonic acid (JA) isolated from Malassezia.

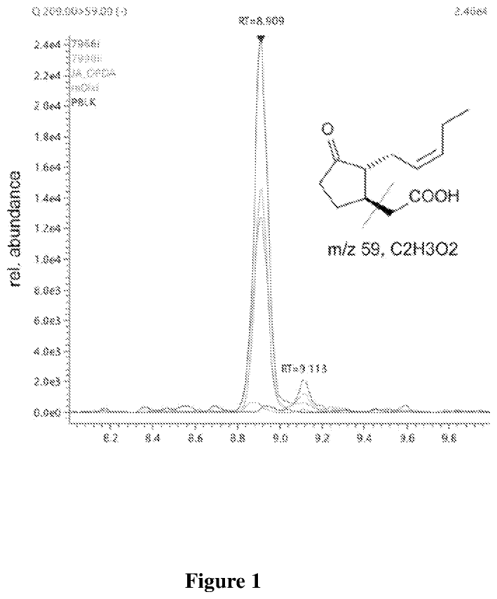

FIG. 2 shows Malassezia JA levels showing inter- and intra-species variability.

FIG. 3 is a heatmap clustering analysis of lipid mediator (oxylipin) profiles from wild-type (WT) M. furfur CBS 14141 and PKS-deletion (KO) M. furfur strains. The three columns on the left indicate gene expression in the WT strain while the three columns on the right indicate gene expression in the KO strains. The boxed sections indicate increased levels of oxylipin species, while the unboxed sections indicate decreased levels of oxylipin species. Increased JA levels in absence of PKS in the KO strain is highlighted by the lighter-colored box.

FIG. 4 shows quantification of JA levels in WT and PKS-KO strains. All data are mean±SE of n=3.

### DETAILED DESCRIPTION

The present specification teaches a method of producing a Jasmonate using a fungal cell, the method comprising a) culturing a fungal cell under conditions suitable for producing the Jasmonate, wherein expression of the polyketide synthase (PKS) gene has been disrupted in the fungal cell, and b) isolating a Jasmonate produced from the fungal cell. The Jasmonate can be secreted by the fungal cell and can be isolated from the cell culture medium or from the gaseous phase of the cell culture system during or after culturing the fungal cell. Suitable fungal cells contain the PKS gene and are capable of producing a Jasmonate.

An “oxylipin” of the present disclosure is a biologically active, oxygenated derivative of a polyunsaturated fatty acid, formed by oxidative metabolism of that fatty acid. As used herein a “jasmonate” is a member of a family of oxylipins which is derived from or related to jasmonic acid (JA). Jasmonates include but are not limited to: jasmonic acid (JA), methyl jasmonate, 7-iso-jasmonic acid, 9,10-dihydrojasmonic acid, 2,3-didehydrojasmonic acid, 3,4-didehydrojasmonic acid, 3,7-didehydrojasmonic acid, 4,5-didehydrojasmonic acid, 4,5-didehydro-7-iso-jasmonic acid, cucurbic acid, 6-epi-cucurbic acid, 6-epi-cucurbic-acid-lactone, 12-hydroxy-jasmonic acid, 12-hydroxy-jasmonic-acid-lactone, 11-hydroxy-jasmonic acid, 8-hydroxy-jasmonic acid, homo-jasmonic acid, dihomo-jasmonic acid, 11-hydroxy-dihomo-jasmonic acid, 8-hydroxy-dihomo-jasmonic acid, tuberonic acid, tuberonic acid-O-beta-glucopyranoside, cucurbic acid-O-beta-glucopyranoside, 5,6-didehydrojasmonic acid, 6,7-didehydrojasmonic acid, 7,8-didehydrojasmonic acid, methyldihydroisojasmonate, amino acid conjugates of jasmonic acid, and the lower alkyl esters, salts, and stereoisomers thereof.

Fungi, including skin resident Malassezia yeasts, can produce JA, but while plant pathways leading to JA production are somewhat characterized, details about JA synthesis in fungi are scarce. For example, in plants, JA biosynthesis through peroxidation of a-linolenic acid by 13-lipoxygenase takes place in the plastid, an organelle not present in fungi. Interestingly, fungal plant pathogens increase their virulence by downregulating plant host defense mechanisms on a metabolic level by secretion of JAs.

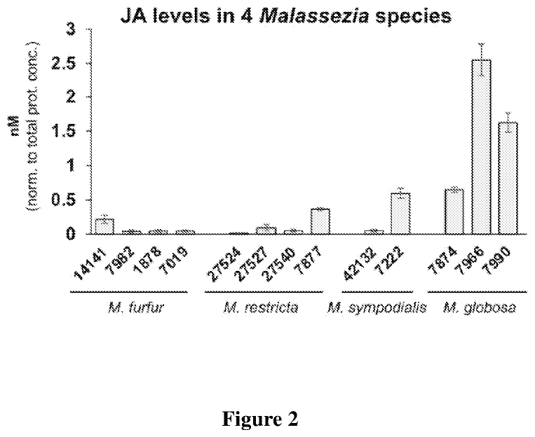

Malassezia is a fungal genus and the main eukaryotic member of the skin microbiome. The inventors have discovered that multiple Malassezia species are able to produce JA (FIG. 2). Besides JA, Malassezia also produce dozens of other oxylipins when cultured in vitro, many of which can be detected on human skin. Jasmonates may influence human skin immunology and hence human health and disease, similar to their immunomodulatory function in plants.

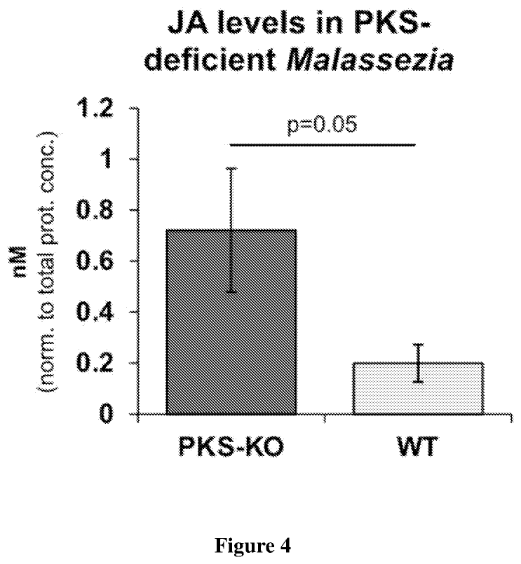

The inventors have discovered that jasmonate production in Malassezia can be enhanced by altering the activity of certain genes involved in fatty acid metabolism. In particular, the inventors have discovered that disrupting the expression of the polyketide synthase (PKS) gene can increase JA production in Malassezia by several fold (FIG. 4).

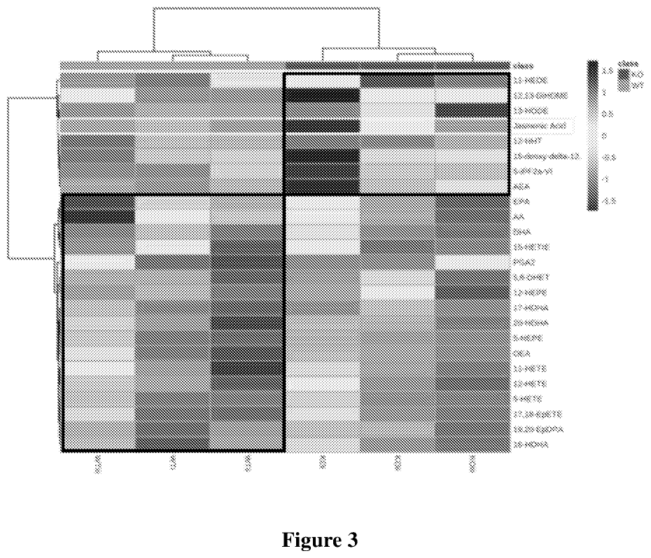

Polyketide synthase is a multi-domain enzyme complex involved in the biosynthesis of polyketides, a large class of secondary metabolites in bacteria, fungi, plants and a few animal lineages. Biosynthesis of polyketides share similarities with fatty acid biosynthesis, which is the pathway responsible for oxylipin and jasmonate production. The inventors have discovered that disruption of PKS expression in Malassezia leads to increased biosynthesis of certain oxylipins, including JA, at the expense of other oxylipins, including the polyunsaturated fatty acids (PUFAs) arachidonic acid (AA), eicosapentaenoic acid (EPA), and docosahexaenoic acid (DHA) (FIG. 3). Without wishing to be bound by theory, this may indicate that in Malassezia the PKS complex is not directly involved in the synthesis of Jasmonate-family oxylipins, but Jasmonate production is limited by competition with other PUFA pathways. Loss of PKS-mediated PUFA and oxylipin biosynthesis through disruption of the PKS gene may remove substrate competition and/or stimulate the biosynthetic pathways involved in production of Jasmonic acid and other oxylipin subgroups in Malassezia.

Accordingly, in some embodiments of the method herein, the fungal cell for producing the jasmonate is Malassezia spp., wherein the expression of the polyketide synthase (PKS) gene has been disrupted in the Malassezia cell. Reference to a Malassezia species in this disclosure includes all strains, isolates and serotypes classified under that species, and further includes any new species which may be classified under the Malassezia genus, and any species which may be reclassified under the Malassezia genus. The particular Malassezia species is not limited and may be appropriately selected depending on its capacity for Jasmonate production. In preferred embodiments, the Malassezia cell produces one or more Jasmonates endogenously, e.g., through a native metabolic pathway. The Malassezia cell may also be engineered to produce one or more Jasmonates, e.g., through the introduction of genes coding for proteins involved in a Jasmonate-producing metabolic pathway.

Non-limiting examples of appropriate Malassezia species include M. arunalokei, M. dermatis, M. furfur, M. globosa, M. japonica, M. obtusa, M. ochoterenai, M. pachydermatis, M. restricta, M. sympodialis, M. tropica, M. yamatoensis, and the like. Two or more strains may be co-cultured for jasmonate production. The Malassezia yeast may be isolated and identified from a sample (e.g., a skin sample from a human or animal), or it may be obtained from a cell depository (e.g., the American Type Culture Collection (ATCC)) or a provider of biological genetic resources.

In some embodiments, the fungal cell is Malassezia furfur, Malassezia sympodialis, Malassezia pachydermatis, Malassezia japonica, Malassezia yamtoensis and/or Malassezia dermatis. In one embodiment the fungal cell is a strain or isolate of Malassezia furfur, e.g., Malassezia furfur CBS 14141.

A polyketide synthase (PKS) of this disclosure includes any one of a family of enzymes which catalyse the formation of polyketide compounds, and is encoded by a PKS gene. The PKS enzyme may be a known or naturally occurring PKS, or a polypeptide homologous thereto or derived therefrom and exhibiting one or more enzymatic activities characteristic of polyketide synthases. A PKS herein may be a Type I, Type II or Type III PKS. The PKS is encoded by a single gene or by a gene cluster.

The term “expression” refers to the transcription and/or translation of a particular nucleotide sequence driven by a promoter.

The term “encode” or “encoding” includes reference to nucleotides and/or amino acids that correspond to other nucleotides or amino acids in the transcriptional and/or translational sense.

The terms “disruption” and “disrupted” are used interchangeably herein to refer to any genetic or cellular modification that decreases or eliminates expression and/or functional activity of a nucleic acid or an expression product thereof of the polyketide synthase (PKS) gene. For example, disruption of the PKS gene includes within its scope any genetic modification, whether naturally occurring or engineered, that decreases or eliminates expression of the PKS gene and/or the functional activity of a corresponding gene product (e.g. mRNA and/or protein). Genetic modifications include complete or partial inactivation, suppression, deletion, interruption, blockage, or down-regulation of a nucleic acid (e.g., a gene). The genetic modification may be accomplished by forced evolution, random mutagenesis, or more targeted genetic engineering methods, followed by appropriate selection or screening to identify the desired mutants. Illustrative genetic modifications include, but are not limited to, gene knockout, inactivation, or mutation (e.g., insertion, deletion, point, or frameshift mutations that disrupt the expression or activity of the gene product). Gene disruption also includes the use of inhibitory nucleic acids (e.g., inhibitory RNAs such as sense or antisense RNAs, molecules that mediate RNA interference such as siRNA, shRNA, miRNA; etc.), inhibitory polypeptides (e.g., antibodies, polypeptide-binding partners, dominant negative polypeptides, enzymes etc.) or any other molecule that inhibits the activity of the gene or level or functional activity of an expression product of the gene.

In some embodiments, the disruption of the PKS gene in a fungal cell is inheritable, i.e., a disrupted PKS gene in a parental fungal cell can be transmitted to all subsequent generations of daughter cells derived from that fungal cell.

In one embodiment, there is provided a fungal cell that has been modified such that the expression and/or function of PKS has been reduced or eliminated. The fungal cell may, for example, be modified to obtain a PKS knock-out or knock down. The term “knock-out” may refer to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant sequence. On the other hand, the term “knock-down” may refer to reduction in the expression of a gene or its gene product(s). As a result of a gene knock-down, the protein activity or function may be attenuated or the protein levels may be reduced or eliminated.

In some embodiments, the PKS gene has been disrupted by a deletion or an insertion in the gene sequence.

In some embodiments the PKS gene of the fungal cell has been disrupted by nuclease-mediated gene editing. It is known in the art that it is possible to use a site-specific nuclease to make a DNA break in the genome of a living cell, and that such a DNA break can result in permanent modification of the genome via mutagenic non-homologous end joining (NHEJ) repair or via homologous recombination with an exogenous DNA sequence. NHEJ can produce mutagenesis at the cleavage site, resulting in inactivation of the allele. NHEJ-associated mutagenesis may inactivate an allele via generation of early stop codons, frameshift mutations producing aberrant non-functional proteins, or could trigger mechanisms such as nonsense-mediated mRNA decay. The use of nucleases to induce mutagenesis via NHEJ can be used to target a specific mutation or a sequence present in a wild-type allele. The use of nucleases to induce a double-strand break in a target locus is known to stimulate homologous direct repair (HDR), particularly of transgenic DNA sequences flanked by sequences that are homologous to the genomic target. In this manner, exogenous nucleic acid sequences can be inserted into a target locus. Such exogenous nucleic acids can encode any sequence of interest.

In different embodiments, a variety of different types of nucleases are useful for disrupting the PKS gene. In one embodiment, the gene is disrupted using recombinant meganucleases. In another embodiment, the gene is disrupted using a CRISPR nuclease. Methods for making CRISPRs that recognize pre-determined DNA sites are known in the art. In another embodiment, the gene is disrupted using a zinc finger nuclease (ZFN). In another embodiment, the gene is disrupted using transcription activator-like effector nucleases (TALENs) or Compact TALENs. In a further embodiment, the gene is disrupted using megaTALs. In yet another embodiment, the gene is disrupted using an ARCUS nuclease.

In various embodiments, a homing endonuclease or meganuclease is engineered to bind to, and to introduce single-stranded nicks or double-strand breaks (DSBs) in the PKS gene. “Homing endonuclease” and “meganuclease” are used interchangeably and refer to naturally-occurring nucleases or engineered meganucleases that recognize 12-45 base-pair cleavage sites and are commonly grouped into five families based on sequence and structure motifs: LAGLIDADG, GIY-YIG, HNH, His-Cys box, and PD-(D/E) XK. Non-limiting examples of meganucleases include I-Scel (from Saccharomyces cerevisiae), I-CreI (from Chlamydomonas reinhardtii) and I-DmoI (from Desulfurococcus mobilis). The homing endonuclease variant may be designed and/or modified from a naturally occurring homing endonuclease or from another homing endonuclease variant. Homing endonuclease variants may further comprise one or more additional functional domains, e.g., an end-processing enzymatic domain of an end-processing enzyme that exhibits 5-3′ exonuclease, 5-3′ alkaline exonuclease, 3-5′exonuclease (e.g., Trex2), 5′ flap endonuclease, helicase or template-independent DNA polymerases activity.

Illustrative examples of LAGLIDADG homing endonucleases include, but are not limited to: I-CreI, I-Scel, I-AabMI, I-AaeMI, I-Anil, I-ApaMI, I-CapIII, I-CapIV, I-CkaMI, I-CpaMI, I-CpaMII, I-CpaMIII, I-CpaMIV, I-CpaMV, I-CpaV, I-CraMI, I-EjeMI, I-GpeMI, I-Gpil, I-GzeMI, I-GzeMII, I-GzeMIII, I-HjeMI, I-Ltrll, I-Ltrl, I-LtrWI, I-MpeMI, I-MveMI, I-Ncrll, I-Ncrl, I-NcrMI, I-OheMI, I-Onul, I-OsoMI, I-OsoMII, I-OsoMIII, I-OsoMIV, I-PanMI, I-PanMII, I-PanMIII, I-PnoMI, I-ScuMI, I-SmaMI, I-SscMI, and I-Vdil41I.

Some embodiments herein contemplate a megaTAL nuclease that binds to and cleaves a target region of one or more target sites. A “megaTAL” refers to an engineered nuclease comprising an engineered TALE DNA binding domain and an engineered meganuclease, and optionally comprise one or more linkers.

A “TALE DNA binding domain” is the DNA binding portion of transcription activator-like effectors (TALE or TAL-effectors). TALE DNA binding domains contemplated in particular embodiments are engineered de novo or from naturally occurring TALEs, e.g., AvrBs3 from Xanthomonas campestris pv. vesicatoria, Xanthomonas gardneri, Xanthomonas translucens, Xanthomonas axonopodis, Xanthomonas perforans, Xanthomonas alfalfa, Xanthomonas citri, Xanthomonas euvesicatoria, and Xanthomonas oryzae and brgl 1 and hpx17 from Ralstonia solanacearum.

In particular embodiments, a megaTAL comprises a TALE DNA binding domain comprising one or more repeat units that are involved in binding of the TALE DNA binding domain to its corresponding target DNA sequence. A single “repeat unit” (also referred to as a “repeat”) is typically 33-35 amino acids in length. Each TALE DNA binding domain repeat unit includes 1 or 2 DNA-binding residues making up the Repeat Variable Di-Residue (RVD), typically at positions 12 and/or 13 of the repeat. The natural (canonical) code for DNA recognition of these TALE DNA binding domains has been determined such that an HD sequence at positions 12 and 13 leads to a binding to cytosine (C), NG binds to T, NI to A, NN binds to G or A, and NG binds to T. In certain embodiments, non-canonical (atypical) RVDs are contemplated.

Illustrative examples of non-canonical RVDs suitable for use in particular megaTALs contemplated in particular embodiments include, but are not limited to HH, KH, NH, NK, NQ, RH, RN, SS, NN, SN, KN for recognition of guanine (G); NI, KI, RI, HI, SI for recognition of adenine (A); NG, HG, KG, RG for recognition of thymine (T); RD, SD, HD, ND, KD, YG for recognition of cytosine (C); NV, HN for recognition of A or G; and H*, HA, KA, N*NA, NC, NS, RA, S*for recognition of A or T or G or C, wherein (*) means that the amino acid at position 13 is absent. In particular embodiments, a megaTAL contemplated herein comprises a TALE DNA binding domain comprising 3 to 30 repeat units.

In particular embodiments, the engineered nuclease is a TALEN. A “TALEN” refers to an engineered nuclease comprising an engineered TALE DNA binding domain and an endonuclease domain (or endonuclease half-domain thereof), and optionally comprise one or more linkers. TALENs contemplated in particular embodiments comprise an N-terminal domain, a TALE DNA binding domain comprising about 3.5 to 30.5 repeat units, e.g., about 3.5, 4.5, 5.5, 6.5, 7.5, 8.5, 9.5, 10.5, 11.5, 12.5, 13.5, 14.5, 15.5, 16.5, 17.5, 18.5, 19.5, 20.5, 21.5, 22.5, 23.5, 24.5, 25.5, 26.5, 27.5, 28.5, 29.5, or 30.5 repeat units, a C-terminal domain, and an endonuclease domain or half-domain. In one embodiment, a TALEN contemplated herein comprises an endonuclease domain of a Type-IIS restriction endonuclease. In one embodiment, the Type-IIS restriction endonuclease is Fok I.

In particular embodiments, the engineered nuclease is a zinc finger nuclease (ZFN). A “ZEN” refers to an engineered nuclease comprising one or more zinc finger DNA binding domains and an endonuclease domain (or endonuclease half-domain thereof), and optionally comprise one or more linkers. In particular embodiments, the ZFN comprises a zinger finger DNA binding domain that has one, two, three, four, five, six, seven, or eight or more zinger finger motifs and an endonuclease domain (or endonuclease half-domain). Typically, a single zinc finger motif is about 30 amino acids in length. Zinc fingers motifs include both canonical C2H2 zinc fingers, and non-canonical zinc fingers such as, for example, C3H zinc fingers and C4 zinc fingers. Zinc finger binding domains can be engineered to bind any DNA sequence.

Individual zinc finger motifs bind to a three or four nucleotide sequence. Candidate zinc finger DNA binding domains for a given 3 bp DNA target sequence have been identified and modular assembly strategies have been devised for linking a plurality of the domains into a multi-finger peptide targeted to the corresponding composite DNA target sequence. Other suitable methods known in the art can also be used to design and construct nucleic acids encoding zinc finger DNA binding domains, e.g., phage display, random mutagenesis, combinatorial libraries, computer/rational design, affinity selection, PCR, cloning from cDNA or genomic libraries, synthetic construction and the like.

In particular embodiments, ZNFs contemplated herein comprise, a zinc finger DNA binding domain comprising two, three, four, five, six, seven or eight or more zinc finger motifs, and an endonuclease domain or half-domain from at least one Type-IIS restriction enzyme. In one embodiment, the endonuclease domain or half-domain is from the Fok I Type-IIS restriction endonuclease.

In various embodiments, a CRISPR (Clustered Regularly Interspaced Short Palindromic Repeats)/Cas (CRISPR Associated) nuclease system is engineered to bind to, and to introduce single-stranded nicks or double-strand breaks (DSBs) in, one or more target sites. The CRISPR/Cas nuclease system is a recently engineered nuclease system based on a bacterial system that can be used for mammalian genome engineering. Engineered endonucleases based on the CRISPR/Cas9 system are also known in the art. A CRISPR endonuclease comprises two components: (1) a caspase effector nuclease, typically microbial Cas9; and (2) a short “guide RNA” comprising a nucleotide targeting sequence that directs the nuclease to a location of interest in the genome.

The term “CRISPR” refers to a caspase-based endonuclease comprising a caspase, such as Cas9, and a guide RNA that directs DNA cleavage of the caspase by hybridizing to a recognition site in the genomic DNA.

In some embodiments a zinc finger protein, a nuclease comprising a Transcription activator-like effector (TALEN) nuclease or a CRISPR/Cas system is used to disrupt the PKR gene.

In some embodiments the CRISPR/Cas system is CRISPR/Cas9. In some embodiments the CRISPR/Cas system comprises at least one nucleic acid encoding a CRISPR nuclease and at least one nuclease acid encoding a guide RNA. The two nucleic acids may be introduced separately, e.g., in different vectors or separate mRNAs, or together, e.g., in a polycistronic construct separated by a viral self-cleaving peptide or an IRES element.

In some embodiments, the CRISPR/Cas system comprises an exogenous sequence for integrating into the yeast genome via homologous recombination following cleavage by the nuclease. The design of exogenous sequences for replacing a native sequence in CRISPR/Cas gene editing is well known in the art.

Selection of successful knock-out or knock-down strains may be performed by introducing a noursothricin (NAT) selection cassette into the fungal cell during gene editing. Nourseothricin (NTC) is a metabolite produced by Streptomyces noursei that belongs to the streptothricin-class aminoglycoside antibiotics that inhibit protein synthesis. NTC N-acetyl transferase (NAT) derived from S. noursei inactivates NTC by acetylating the β-amino group of the β-lysine residue on NTC, thus cells which are successfully gene-edited will contain the NAT resistance cassette and will be able to grow in the presence of NTC.

In some embodiments the expression of the PKS gene is disrupted using an inhibitory nucleic acid or small molecule that inhibits expression of the PKS gene.

In one embodiment, the fungal cell comprises or has been contacted with an inhibitor of PKS. The inhibitor may be, e.g., a small molecule or a polypeptide. Non-limiting examples of small molecule PKS inhibitors include kraussianone 6, kraussianone 1, neodiospyrin, clionamine D, bromotopsentin, isodiospyrin, spongotine A, kraussianone 3, 14-β-hydroxybufa-3,5,20,22-tetraenolide and kraussianone 7.

In another embodiment, the fungal cell comprises or has been contacted with an inhibitory nucleic acid, e.g., an inhibitory RNA such as sense or antisense RNA, or a nucleic acid that mediates RNA interference such as siRNA, shRNA, miRNA. Methods for RNA-mediated gene disruption are well known in the art.

The fungal cell may be cultured in any nutrient medium that supports the growth of the cell and the production of Jasmonates by the cell. Cultivation and fermentative incubation of the fungal strain is generally accomplished in an aqueous medium in the presence of essential nutrient substances (carbon source, nitrogen source, inorganic salts and growth factors). Examples of inorganic salts that can be included in the nutrient medium include, but are not limited to, phosphate and/or sulphate salts of sodium, calcium, magnesium, and potassium. Additional nutrients may also be added, such as one or more B vitamins, one or more trace minerals such as iron, manganese, cobalt, copper, zinc, etc., as familiar to those skilled in the art. Fungal growth hormones such as 10-oxo-trans-8-decenoic acid and hercynine also may be included in the nutrient medium. Media commonly used for fungal culture include, for example, brain-heart infusion medium, Czapek-Dox medium, potato dextrose medium, Sabouraud's heart infusion medium, Sabouraud's dextrose medium, dermatophyte medium and bird seed medium. The inventors have found that Dixon medium and modified Dixon medium, which both contain malt extract, glycerol, desiccated ox-bile, peptone, oleic acid and Tween 40, are particularly suitable for culturing Malassazia. The composition of Dixon medium is provided in the Examples.

In some embodiments, the methods herein comprise providing the fungal cell with a fatty acid source in step a). Sources of fatty acids can include, for example, Tween 20, Tween 40, Tween 60, Tween 80, butyric acid, valeric acid, caproic acid, heptanoic acid, caprylic acid, pelargonic acid, capric acid, undecanoic acid, undecylenic acid, lauric acid, myristic acid, myristoleic acid, palmitic acid, palmitoleic acid, margaric acid, stearic acid, oleic acid, linoleic acid, linolenic acid, arachidonic acid and euricic acid. The fatty acid source may improve fungal growth and/or fungal production of Jasmonates.

In a typical process, the fungal cells are first cultivated in inoculum quantities to produce a mature culture in a nutrient medium. The culture is inoculated into a fermenter nutrient medium and allowed to establish itself. Fermentation is continued until a steady concentration of the Jasmonate product is present.

Culture can be conducted in batch or continuous mode. In batch fermentation, the nutrient medium and culture are combined and fermented until the quantity of the Jasmonate product in the culture is constant. In a continuous process, the nutrient medium may be continuously recirculated through a fermentation reactor, and product may be removed from the recirculating medium.

Malassezia fermentation can be carried out under agitation at about 150 rpm. The cultivation temperature can be about 32° C. Cultivation and incubation can proceed under aerobic conditions at a pH of about 6. The Jasmonate product can be isolated after at least 2 days of culture, or when no additional Jasmonate is produced.

The Jasmonate product can be isolated from the nutrient medium and/or the fungal cell fraction, using any methods known in the art, for example, by liquid-liquid extraction with an extraction solvent such as methanol to form a Jasmonate extract. The extraction solvent can be stripped to provide a concentrated Jasmonate extract. Fractionation can be performed, e.g., with silica gel, to further purify the Jasmonate or to separate different isomers. The Jasmonate product can also be isolated from the gaseous space of a culture or fermentation chamber, for instance, by passing the gases through an adsorbent (e.g., Super-Q® filter trap) and eluting the Jasmonate retained on the adsorbent with an appropriate solvent (e.g., methanol, dichloromethane or ether).

In some embodiments of the method herein, the Jasmonate produced by the yeast is Jasmonic acid, methyl-Jasmonate, or a Jasmonic acid conjugated with an amino acid. Jasmonic acid may be naturally conjugated to any amino acid, e.g., isoleucine, leucine, valine or methionine.

In some embodiments the Jasmonate is secreted by the fungal cell, and step b) comprises isolating the secreted Jasmonate. The Jasmonate may be naturally secreted by the fungal cell, or the fungal cell may be engineered to secrete or improve the secretion of the Jasmonate. The secreted Jasmonate may be isolated from the culture medium or from the airspace above the culture medium.

Also disclosed herein is a method of enhancing the production of a Jasmonate from a fungal cell, the method comprising disrupting expression of the polyketide synthase (PKS) gene in the fungal cell. Suitable fungal cells contain the PKS gene and are capable of producing a Jasmonate. The fungal cell may be a yeast cell, e.g., Malassezia spp.

In some embodiments, the method further comprises culturing the fungal cell under conditions suitable for producing the Jasmonate and isolating the Jasmonate produced from the fungal cell.

Provided herein is an engineered fungal cell for producing a Jasmonate, wherein the polyketide synthase (PKS) gene has been disrupted in the fungal cell. Suitable fungal cells contain the PKS gene and are capable of producing a Jasmonate. The engineered fungal cell may be a yeast cell, e.g., Malassezia spp.

Disclosed herein is a microbiome composition comprising a fungal cell, wherein the polyketide synthase (PKS) gene has been disrupted in the fungal cell. The fungal cell is preferably one that produces a Jasmonate.

In some embodiments, the microbiome composition is a skin microbiome composition. The microbiome composition may comprise the fungal cell and one or more skin commensal microorganisms. By “skin commensal microorganisms” is meant prokaryotes and eukaryotes that live and multiply on skin (preferably human skin) or temporarily inhabit skin (preferably human skin) in vivo. Non-limiting examples of skin commensal microorganisms include Alphaproteobacteria, Betaproteobacteria, Gammaproteobacteria, Actinobacteria, Propionibacteria, Corynebacteria, Clostridiales, Lactobacillus, Staphylococcus, Bacillus, Micrococcus, Streptococcus, Bacteroidales, Flavobacteriales, Enterococcus, Pseudomonas, Malassezia, Maydida, Rhodotorula, Epicoccum and Cryptococcus. The one or more skin commensal microorganisms may help to support or enhance the growth of the fungal cell and/or improve Jasmonate production in the fungal cell.

The microbiome composition may be formulated for topical application. The microbiome composition may be capable of producing a Jasmonate when topically applied. The microbiome composition may comprise one or more dermatologically acceptable carriers which are compatible with the fungal cell and other skin commensal microorganisms. Dermatologically acceptable carriers are carriers which are suitable for application to skin or keratinous tissue without undue toxicity, incompatibility, instability, or allergic response. The dermatologically acceptable carrier may be in a wide variety of forms, such as simple solutions (water-based or oil-based), solid forms (gels or sticks) and emulsions (water-in-oil or oil-in-water). Examples of dermatologically acceptable carriers include, but are not limited to, distilled or deionised water; propylene glycol; glycerol; silicones such as volatile silicones, amino or non-amino silicone gums or oils, and mixtures thereof; mineral oils; plant oils such as olive oil, castor oil, rapeseed oil, coconut oil, wheatgerm oil, avocado oil, macadamia oil, apricot oil, safflower oil, flax oil, tamanu oil, lemon oil and mixtures thereof; waxes; and organic compounds such as C₂-C₁₀ alkanes, acetone, methyl ethyl ketone, volatile C₁-C₁₂ alcohols, esters of C₁-C₂₀ acids and of C₁-C₈ alcohols such as methyl acetate, butyl acetate, ethyl acetate, and isopropyl myristate, dimethoxyethane, diethoxyethane, C₁₀-C₃₀ fatty alcohols such as lauryl alcohol, cetyl alcohol, stearyl alcohol, and behenyl alcohol; C₁₀-C₃₀ fatty acids such as lauric acid and stearic acid; C₁₀-C₃₀ fatty amides such as lauric diethanolamide; C₁₀-C₃₀ fatty alkyl esters such as C₁₀-C₃₀ fatty alkyl benzoates; naturally and synthetic hydrophilic polymers such as hydroxyalkylcellulose, carboxymethylcellulose, polyethylene glycol, polypropylene glycol, polyvinylpyrollidone and polyvinylalcohol; poly(acrylic acid) polymers or co-polymers such as Carbopol® products; and mixtures thereof.

The microbiome composition may further comprise one or more optional components known or otherwise effective for use in cosmetic or skin care products, provided that the optional components are physically and chemically compatible with the microbial component and the carrier component described herein, or do not otherwise unduly impair product stability, aesthetics, or performance. Some non-limiting examples of such optional components include plasticisers, surfactants (which may be anionic, cationic, amphoteric or non-ionic), neutralising agents, emollients, lubricants and penetrants such as various lanolin compounds, vitamins, proteins, preservatives, dyes, tints, antioxidants, reducing agents, sunscreens, thickening agents (e.g., polymeric thickeners, such as xanthan gum), non-polymeric thickeners including clays, and perfume.

Disclosed herein is a microbiome composition as defined herein for use as a cosmetic or as a medicament. Jasmonate production by the fungal cell in the microbiome composition may yield one or more cosmetic and/or pharmacological effects. The cosmetic or pharmacological effect of the Jasmonate may be enhanced by the skin commensal microorganisms in the composition.

The microbiome composition may be a cosmetic composition, i.e., a topically-applied composition which is intended to improve the condition and/or appearance of the skin or keratinous tissue or otherwise provide a skin care benefit. Non-limiting examples of skin care benefits include improving skin appearance by providing a smoother, more even appearance; increasing the thickness of one or more layers of the skin; improving the elasticity of the skin or hair; reducing the oily, shiny, and/or dull appearance of skin or hair; improving the hydration status of the skin or hair; improving the appearance of fine lines and/or wrinkles; improving skin barrier properties; reducing the appearance of redness or skin blotches; and/or improving the brightness, radiancy, or translucency of skin.

A cosmetic composition herein may be in the form of a composition for hair care, in particular a shampoo, a lotion, a cream, a gel or a mousse. It may also be in the form of a composition for cleaning, protecting, treating or caring for the face, the hands, the feet, the large anatomical folds or the body, for example, an ointment, a cream, a sunscreen, a milk, a lotion, a gel, a moisturiser, a body wash, a deodorant, or an aftershave. Alternatively, the composition may be a make-up composition for the body or the face such as a foundation; a composition against insect bites; or an analgesic or anti-pruritic composition.

Alternatively, the microbiome composition may be a dermatological or pharmaceutical composition for use as a medicament for treating certain skin diseases, such as eczema, rosacea, psoriasis, dermatitis, actinic keratosis or severe pruritus. The pharmaceutical composition may also be used as an antiviral, antibacterial, antifungal, anti-aging, anti-inflammatory or analgesic composition, or used to treat autoimmune or neurological diseases, or cancer.

Disclosed herein is the use of a microbiome composition as defined herein for producing a Jasmonate. The microbiome composition may be used for Jasmonate production in culture, e.g., the composition may be cultured in a culture vessel, a fermenter or a bioreactor for Jasmonate production. Alternatively, the microbiome composition may produce Jasmonates when applied as a topical formulation.

Jasmonates produced according to the present methods can be used for various applications in agriculture, food, fragrances, and medicine. For example, methyl jasmonate can be used as a food and flavour ingredient in products such as perfumes, personal care products, household care products, oral consumable products, and so forth.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### Examples

### Methods

Generation of Polyketide Synthase (PKS)-Deficient M. furfur Strains

To genetically modify the commercially available Malassezia furfur strain (CBS 14141), an Agrobacterium-mediated mutagenesis method using CRISPR/Cas9 was applied. The guide RNA sequence (sgRNA) 5′-ATACTTTGAGCTGCTCAAGG-3′(SEQ ID NO: 3) was cloned into a plasmid allowing expression of CRISPR/Cas9 components in Malassezia and allowing the induction of a specific double strand break (DSB) within the coding sequence for Polyketide Synthase (PKS) in M. furfur. To efficiently knock out (KO) the M. furfur PKS gene following induction of a DSB within the PKS coding sequence, a homologous recombination (HR) repair template was generated, containing 1.5 kb flanking arms homologous to sequences up- and down-stream of the PKS coding sequence. The PKS gene is located within CBS 14141 chromosome 1 at the CP046234.1 locus. The nucleic acid sequence of the PKS gene with flanking regions is provided as SEQ ID NO: 1 (in appended sequence listing).

Identification of successful PKS gene KO strains was facilitated by knock in (KI) of a Nourseothricin (NAT) selection cassette in lieu of the PKS gene. The sequence and the location of the NAT selection cassette within the putative PKS gene (CP046234.1 locus) in CBS 14141 chromosome 1 is provided as SEQ ID NO: 2 (in appended sequence listing). Briefly, the sequence from nucleotide position 1,746,826 to 1,751,948 in chromosome 1 of CBS 14141 was replaced with the knocked-in NAT selection cassette sequence.

The HR repair plasmid, Agrobacterium-mediated transformation and selection was generated and performed as previously described. In brief, the plasmid was electroporated into A. tumefaciens EHA105 and successful transformation was verified using PCR and restriction digestion. Log-phase cultures of M. furfur CBS14141 and transformed A. tumefaciens which has been grown in induction medium containing 100 μM acetosyringone (Sigma) were separately harvested. Equal proportions of Malassezia and Agrobacterium cells were mixed thoroughly and passed through 0.45 μm mixed cellulose membrane (Merck, Millipore) and transferred onto induction media agar supplemented with 200 μM acetosyringone. Cells were incubated at room temperature for 5 days before the cells were washed in 20 mL sterile PBS and transferred onto mDixon with 100 μg/mL NAT, 200 μg/mL cefotaxime, and 10 μg/mL tetracycline.

### Growth Conditions for In Vitro Production of Jasmonates by Malassezia

All Malassezia strains were cultured in modified Dixon media (36 g/L malt extract, 2 mL/L glycerol, 20 g/L desiccated ox-bile, 6 g/L peptone, 2 mL/L oleic acid, 10 mL/L Tween 40, and pH 6), and incubated in a rotary shaker at 32° C. and 150 rpm. 15 ml of triplicate cultures in late exponential growth phase were harvested by centrifugation, washed three times in PBS, transferred to 2 ml Eppendorf tubes and stored at −80° C. prior to Jasmonate extraction.

### Measurement of Jasmonates in Malassezia Culture

JA levels in culture medium was monitored by extraction with methanol:water: acetic acid (20:80:0.02 by v/v), spiked with JA-d4 as the deuterated internal standard, and enriched using Strata-X 33 mm polymeric solid reversed phase (SPE) extraction columns. Extracted samples were then analyzed using reversed phase high-performance liquid chromatography (HPLC), coupled to triple-quad mass spectrometry (MS) analysis.

Reversed phase separation was performed on a Phenomenex, Kinetex C8 (2.1× 100 mm I.D×150 mm L., 2.6 μm) column and maintained at 40° C. The mobile phase consisted of (A) water/formic acid (100/0.1, v/v) and (B) ACN. The stepwise gradient conditions were carried out for 30 min as follows: 0 min, 10% of solvent B; 0-5 min, 10-25% of solvent B; 5-10 min, 25-35% of solvent B; 10-20 min, 35-75% of solvent B; 20-20.1 min, 75-98% of solvent B; 20.1-28 min, 98% of solvent B; 28-28.1 min, 98-10% of solvent B; and final 28.1-30 min, 10% of solvent B. The flow rate was 0.4 mL/min, injection volume was 10 μL, and all samples were maintained at 4° C. throughout the analysis.

A mixture of representative native and internal standards was injected and run with the column to optimize the source parameters. The electrospray ionization was conducted in positive mode. Drying gas temperature was set at 270° C. with a gas flow of 10 L/min. Sheet gas temperature was set at 250° C. with a gas flow of 10 L/min. The nebulizer gas flow was 230 kPa. The dynamic MRM option was used and performed for all compounds with optimized transitions and collision energies. MRM transitions (precursor and product ions) and collision voltages were as follows: cis-OPDA (293→81.15;−30 eV), Jasmonic acid (211.1→133.15; −13 eV). The determination and integration of all peaks was manually performed using the LabSolutions Insight software. Peaks were smoothed before integration and peak to peak Signal/Noise ratios were determined using the area under the peaks.

### Example 1: Malassezia Spp. Produce Jasmonates (JA) In Vitro

It has been shown that Malassezia produces at least 46 oxylipins when cultured in vitro, many of which are detected on human skin. Furthermore, several fungal species, including skin resident Malassezia yeasts (see FIG. 2) produce JA. Methodologies to cultivate and produce JA in vitro have been established (see Methods above). But while plant pathways leading to JA production are somewhat characterized, details about JA synthesis in fungi are scarce. Manipulating biosynthetic pathways involved in production of JA in Malassezia may significantly increase yields. In vitro cultured Malassezia globosa species produced more JA than the M. furfur species (see FIG. 2), and as such can be a more promising basis for exploring JA yield improvement through genetic manipulation.

### Example 2: CRISPR/Cas9-Mediated Knockout (KO) of Polyketide Synthase (PKS) in M. furfur Significantly Increases JA Production

Malassezia yeasts are not capable of de novo producing polyunsaturated fatty acids (PUFAs) given the lack of the required 49-desaturase. However, Malassezia has been shown to accumulate several PUFAs, including «-linolenic acid (ALA), the substrate for JA synthesis, suggesting that alternative pathways for PUFA production in Malassezia may exist. Interestingly, under certain conditions, PKS has been shown to produce PUFAs both in prokaryotes and eukaryotes. PKS activity was manipulated through genetic engineering in M. furfur to investigate it this may affect PUFA and consequently JA levels. Interestingly, CRISPR/Cas9-mediated deletion of the gene encoding Polyketide Synthase (PKS), a multi-domain enzyme complex involved in the production of related chemical structures resulted in significantly (3.6-fold) increased biosynthesis of certain oxylipins, including Jasmonic acid (FIGS. 3 and 4) commensurate with reduction of similar oxylipins, including the PUFA EPA, AA and DHA. The use of CRISPR/Cas9-technology to knock out PKS activity in Malassezia furfur thus generates a unique microbial strain with improved capabilities for enhanced JA biosynthesis.

Surprisingly, deletion of the PKS gene in M. furfur did not reduce JA levels but instead significantly enhanced JA production compared to the wild-type strain. This indicates that in Malassezia the PKS complex is not directly involved in Jasmonate-family oxylipins, but their production is limited by competition with other PUFA pathways. Loss of PKS-mediated PUFA and oxylipin biosynthesis by the M. furfur mutant strain may remove substrate competition and/or stimulate the biosynthetic pathways involved in production of Jasmonic acid and other preferred oxylipin subgroups. This data also proves that deletion of the PKS gene from other, higher producing strain such as Malassezia globosa would increase their Jasmonate production.

It will be appreciated that many further modifications and permutations of various aspects of the described embodiments are possible. Accordingly, the described aspects are intended to embrace all such alterations, modifications, and variations that fall within the spirit and scope of the appended claims.

1. A method of producing a Jasmonate using a fungal cell, the method comprising a) culturing a fungal cell under conditions suitable for producing the Jasmonate, wherein expression of the polyketide synthase (PKS) gene has been disrupted in the fungal cell, and b) isolating a Jasmonate produced from the fungal cell.

2. The method of claim 1, wherein the method comprises disrupting the PKS gene prior to step a).

3. The method of claim 1, wherein the method comprises providing the fungal cell with a fatty acid source in step a).

4. The method of claim 1, wherein the fungal cell is a yeast cell.

5. The method of claim 4, wherein the yeast cell is Malassezia spp.

6. The method of claim 1, wherein the PKS gene has been disrupted by a deletion or an insertion.

7. The method of claim 1, wherein the PKS gene of the fungal cell has been disrupted by nuclease-mediated gene editing.

8. The method of claim 7, wherein the nuclease is a zinc finger protein, a nuclease comprising a Transcription activator-like effector (TALEN) nuclease or a CRISPR/Cas system.

9. The method of claim 8, wherein the CRISPR/Cas system is CRISPR/Cas9.

10. The method of claim 8, wherein the CRISPR/Cas system comprises at least one nucleic acid encoding a CRISPR nuclease and at least one nuclease acid encoding a guide RNA.

11. The method of claim 8, wherein the CRISPR/Cas system comprises an exogenous sequence for integrating into the yeast genome via homologous recombination following cleavage by the nuclease.

12. The method of claim 1, wherein expression of the PKS gene is disrupted using an inhibitory nucleic acid or small molecule that inhibits expression of the PKS gene.

13. The method of claim 1, wherein the Jasmonate is Jasmonic acid, methyl-Jasmonate, or a Jasmonic acid conjugated with an amino acid.

14. The method of claim 1, wherein the Jasmonate is secreted by the fungal cell, and step b) comprises isolating the secreted Jasmonate.

15. A method of enhancing the production of a Jasmonate from a fungal cell, the method comprising disrupting expression of the polyketide synthase (PKS) gene in the fungal cell.

16. The method of claim 15, wherein the method further comprises culturing the fungal cell under conditions suitable for producing the Jasmonate and isolating the Jasmonate produced from the fungal cell.

17-21. (canceled)


SEQUENCE LISTING (3 sequences)

SEQ ID NO: 1 - DNA, 8523 bp, Malassezia furfur
  source 1..8523: mol_type = genomic DNA
acttttgtctgacgcgccgttcatattctccgctggttcggttgtatactcattctatcaatcccttcgcgttgtgtttttgaagtgagcgccacacttctttcattcaacgcttgcacctgcatcttccttttccatttcccgcaatatgtctagcactacgcccaaccagcaacagcagtcgcaaggccaggacgaacgcccgcctagcatctcggtggtcggtatcggcctccgtcttcctggagcacgcgacaagaaggaatactttgagctgctcaaggagggccgctgtgccattggccgtgttccggccgaccgttggaacgctgaggcgatgactcaggacggtggtgttcctggcaaggtgatcacggaccagggtggtttcgttgacccccacacggacgttgatacgctcgagtttggtatctcgcccgccgaggcgcgccagatcgacccgcaccaagtcgtgctggttgagtgcgtgtaccaggcactcgaggactcgggtatcaactaccgtggtaccaacaccggtgtgtacgtcacgggttcgcccgacgtgcacaacctgggtaaggacatgtacgacatgggtccctacagtgcgaccggtgcggccttctcgatgcaggccaaccgtctgtcgtacctgttcgacctgcgtggtccgtcgatctacctcgacacggcttgctcgtcgagtatcacgtgtctgcaccttgcgcgcacggccatcctgcgcggcgactgtgacatggcggtcgtggcggcggtcaacctgattcttgcgccggatgcctcgctgtcgttctcgacgctgggcaccctgtccaagacgggtctctgccacacgttcgacgcgtcggccgacggttacgtccgtggtgagggctgcacggtcattgtgctgcagcgcaccgaggacgccgtgcgcctgggcagccacgtgtactcggagatcactggtacgcacatcaatgcaaacggcaagggcaagagtatcactctgcccgacggcccgcagcagaaggccgctacgatcgccgcctacaaggaggcgaagcgcgacttcagcgagacggtgtacgtcgagtgccacggtacgggtacgccggtcggtgacccgatcgaggcgaacgccgtcggtcaggtcttctcgcctggccgctcgagcgacgacaagctgcgcattggttcgtccaagaccaatgtcggtcacttggagcctgctgcgggtcttgtgggtctgatcaagaactgctacaccctcgacacgggctacctgctgccgcacctgcacttccagacgccgagccccaagatccactgggacgacttccagattcaggtgcagaccgcctatgagccgctccccgagaagaagctgagcaaggacggcaagtttatcgtgtcgctctcctcgttcggtttcggtggtgccaactcgcacacggtcatggagcgtgtgccgaacaaggcgcgcattgagaacttcaaggcgcccgcggcgacggacccgatgcttgtcgcggtgggtgcgctgtcgaaccgtgcggtgacctcgctcgagaactcgatggaggagttctggggcaagtcgccgaacccgatgcactcggagctcatggcccgtaccattacggagcgtgcgcgcggtaaccccaacatggcgtttgccgtggcgtcgcccaacgagccgctgcagttcaacgagacgaccgccacctcgtcgcccgacttcaacccgctcaaggcgtttgtgttctgtggtcagggtccccagcacgaggacatgggtcgccacctgtacgcgcgctttggtgcgttccgtgactcggtgcacaagtcgttcgaggtggtgcagcgctaccacaagggcgagtttgtcaacagcggtctgttctcctcggacaaggacgccaacgtgcagaagacgcagtcgggtcactggtcggtcgaggcgattgtgattggtatcaccatcttccagatcgccctgtacgacctgtgggtgtcgctgaacgtggtgcccgacgtggtcatgggccactcggtcggtgagattgctgcgatgtacgcctcgggtgccttctcgcacgagaaggcgatccgcacggccattgcgcgctccaacgccctcggcctgctcgacacggtcgacggtacgatggccgcgctcggcatcagccgcgaggaggcgcagacgatcctcgacgaggtcctgaaggagaacaacgtgaccaccggtctgtgggtctcggcgagcaactcgaccaaggccgtgtcggtgtcgggtgagacgaagctcgtgcgcgaggtcgtcaagaagtgcgaggctgcgggcatctttgcgcgtgagctgcgtgtcggtggtccgtaccactcgccgatggtgtcgccctgcaaggagcccttcctgtcggaggtgtacccgctggtcgatgccgagaagaacgtgccgacgcgccgcttcatctcgacggtgcacggccgcatgcacgaggtcggcgagtcgcttgacgccgagtactgctgggacaatgtcagctcgcctgtgctgttccgcgagtcgatcgaggcgcttgagcagtaccgcaaggagcaggaccgtggcctgctgatcgtcgagattgcgccgcactcggtgcttggcgcgtacatggacgagatcacgcgcgccatcgacgccgagcgcacgacgattgtgtcgtcggcgcgccgccccaacttcaagaagggcgagacgcgcgacacccccggcgaggttacccagttcctcaccgctgtcggccagtcgatgcaggcgggcgagcgcaacatgatcgtgtacaagctgtactcggacgactttgtcgactgcccgctcggcatcaactcggagctgccggagctgccgccgtaccccttcctgccgaagccgaagcgcacgaccgagatcagcttccccgagcacatccgctgccgcccgccgggcccgccgctgtgcagcgagatgctccgtatcaatgcgcagacgcacagctggaccaagggccacaagatccgtggcacgatcattttcccgggtgccggttacgccgagtcggccctcgaggccggcgcgcgcaccatcaccgacatggacatccaccgtgcgtttgtcattgaggaggagggccagccgaagtacgcccgctttgtcgagacgggcatcaacggcggctgggagttccgctcgtcgggcaagaacgcggtcaacgacggtggtatcatctacgaccagctgcacgcctcgggccacatgtcgcccgagtacacgcgtgtcggccccgactcgctccaggaggtgtttggcgagaactggctcgagcactttgacattatcatggacggccagaccttctacgaccgcctgaagcccaacggttcgcagcacacgggtgcgttcggtatgatcaacgaggtgcgtggctcgacgacgcgcgaggacgactaccttgcgtttgtcgacgtgctgcccgacctctggacgcaccccgagtcgcagcacatggtgttccaccccggtctgctcgactcggcgttcctgtgcacgtggattcccgcgctgccgttcgacggcagcaagctcggtgtcgacgccttcctgccgaaccacctcgacatcctcgcgatgcgcgcgacgcccgaggaggtgcgtgcggcgaagcagattgtactctacgtccagacgctcatctcgaacgaccaccacatcaagcacaacattctgatgtttgaccgcaagacgggcaagacgctcgcgttcctgcgtggtctcgagtgcacgcgtatcaaggacacggtcaaggagcgcgaggcgtacaccgagtcgtgggagccgcgtgcgtttgacgccacggctgcgatcgacgaggagtaccgcctgcagccgcagctctcgagcgcctttgtgcaggacatggccgcccacgacgaggaggacgcggtcgccgaggcgctccagcgcggcagtgctgcggcggtgtgcgccgacggcctgcaggaccgcgtgtttgacctgcccggcgtcagcgccgaggtcagcgagagcatcgtcgaggccgcccaggcgctcatctcgtcgtcgctgcagcaggagccgcgccgcgtgttccgcgtgctcgagatgtacgcgcgccgccgcaaggtcaacctgcagccgctcgtggcgtgggcgcagaagcgcggcctgtacgtcgacctggtgcgcctgaacgtcgccaacccggtcaagggcacggatgccttcttcacgggcccgtacgatgtgcccgcggcgattgccgacttccagcgcacgatccactcgacgtttgacctggtcgtgtcgagcgacctgctgcgctcggtgccggacgcctcggcggtggtcaaggccgcggacgacctgctggtccccggcggctacgccgtgtttgccgaggtgaaccctgcggccaaggcgctcgcgcagctcttctacacgagcaaccccgtgtcgctcgaggcgctcggcctgacgccgttcagcaaggcgagcccgacgctgaacgtcgcctacctgcgccgcggcgacaacgcctggccgagcttcccgctgccctcggtgatgcctgccgaggagccgcgcagctcgggccgcttccgccgcgtgaacgaccatgtgctcgcgtactaccacgagcccgacaccgagtacgaggtggtcgaggcggtgcgcgaggcgtttggcgagtcgcccatgggccagctgtgggtccttgccgacgactcggccgacggtgcgcgtggtctgggtctggccggtacgatcaccaacgagattccgacgctcaagtcggtctacgtggcgttcgacaaggcctacacgcgcggccagcgcgatgcgatcctcgaccacctgctcgagcacgaggtcaaggacggcagcgtcgaggtgttcaacatcatccgcaacggccgcctgtacggccgccgtctcgtgaagcagaccgtgatgcaccccaccgagttccagggccaggactgggtgctcgagctcatcgagggccagccggcgagcgtgcaggccattgcgccgcacacgcactacccgccgcacctcggcccgcacgacgtgctcgtccagaccgacgccgtggcgctcaacttcaagaacattctgtccgccaccggtctgctgcctgccaccgaccgcctgtcggagttcggtggtgtggtcaaggccatcggcaaggatgtgcaccgcgtcgcggtcggcgaccgtgtgatgggtacctcgggcggtgtccgtgagggtaccgttgcgatggcctcggagtacgccatcaccaagatccccgacaacatgaccaacaaggaggccgccgcgtacccgatcgcgtacggcaccgtgtggtacggtctggtgcaccttgcgcagatccgcaagggcgagacgatcctcatccacgctgccgccggtggtgtgggtctgtgcgcgatccagattgcgcagcgcaagggcctcgaggtgttctgcacggtgtcgtcgcaggagaagcgcgactacctgcacaacaacctcggtgttccgtacgagaacatggccaactcgcgcagcattgccgagtggcagcaggccggcttcaagtggcttgccaagcgcggcaagacgggcttcgacgtggtgctcaactcgctgcagggcgctgcgctccaggccggtatcgagtcgctcggctacctcggccgctttgtcgacatctcgaagcgtgaccaccttgcgggcaaccccatgtcgatggccggctttgctaaggcgatcaactacatcgccgtcgagctcggtctgctcggtgtgcacgcgcccgagcgcatgggcgcgctcctcgacgagattgccgccgagcacgcccgcgagccgttccgctgcctgatcggccacgagttcgagggcgccaacggcctggtcgagtcgtaccgcctgatggagtcgggcaagcacatcggcaagatcgtcacggacctctcgtccgagattggccccgacgcgcgcaccaagctgtggaacgcgtacaaggtgtacgacccgcgcaagtcgtacgtgctgatcggtggctgtggtggtctcggcccgcgtctgacgcaattcctcatgtacctcggtgcgcgcaacattgtgctcaccggtcgtcgcggccagatctcgcgttcggaccgcctggcgctcgagcgtctggtgcgtgaccccgcgttccccgacgtgacgatcaaggtcatggctgccgacgcgctcaacccgtcggcgatgaacgacgtgtttgatacggcgcgcagccttggcccgatcggcggtgtcttcctcatgtcggtcgtcctgcgcgacgaccagttcatgaagatggacaaggagaagttcgacacggtcgtcaagtccaagatcggcgcactcaacgtcgtgcgcgagctgatcgacatccagacgctcgacttcctcttcctgttctcgtcgaccgccgcgctcttcttcaaccctggtcaggcgaactacaacgcggcgcaggcgtacttcaaccgcttcgcgcaggagaacaagaacgtcatctcgtacgccgtgcccgccatttcggacattggtgtgtacgcccagatgcgctccaagtcgaacaacgccgcgctcaaggtcatggacgccctcgcgtgcaactcgcgtgagctgtgcacgtcgattgcgcagtcgatcgcgcgtctcgtgatgggcggcaccaaccacgctggctactttatccagccgatggactggaaggtcatgcacgagatctcgcctgcgaacctctggtccatttcgcacctggtcgagcactacgacgatgccgacaacgacggtgcggacggcgaggcggaggctgaccccgtcggcacgctcctgggcaagctgctcaacgtcgacatcgagacggtggacgacaccatgttcctgtccacgctcggtctcgactcgctctcggcctcgaagctgagcgcgatcctcatgagcgagtttggcgcgaccgtcacgcagctgcagctgctcggccccgtgtcgattgctgcgctgcgccagattgtcgccgacgccatcgccgagaaggagggtggcgttcccgagaagggcaacacgggcgatgcgcccgccaagaagacggccgccgcccctggcggtgcgtcggcctcgggtgcggcgccgaccagcaaggtcactgcgccgctcgccgagttcgactacgcctcggaggtcgacaagctcgacgacctgcccaagacggtcgcgcacctgcagccgtttgacccggcgatcctcgagccgagcaagaagcaggaggtgctggtgaccggcgccacgggcttcctgggcagctcggcgctcgaggcggtgctcgaggcgctccccaacgcgcacgtcaccgccctggtccgcggctcgcctgccgagggcctgcagcgcatcaagcagatggccgagaagcgcgagctcaagtcgctcaagtcgatggaccgtgtgtcggtcgtgtcgggcgacgtgagccagccgcacctcggcctgtccgaggaggactggacgaagctcgccgagacggttgacctgatcgtgcacgcgagcggcaaggccgaccacctgatcggctaccagggcatcgcccccgtcaacacctactcgacgcgcgaggtgctgcgcctggcgtcggacaagaaggtcaaggcggtcgccaacattggctcgaccaacatgtggctcagcttcaccgcggagcccaagacggacgaggtggtgatggaggacctcgacctggacacgctcaagacgggcctgttcaacggctactcgcagtccaagtgggtgtcggagatgctcacgcgccgcatgcacgagcgcggtgtgcccgccatgacggtgcgccccggtgtgctcggcggtagcgcctcgggccgctaccaccccaacgaggacagcttcctgtggcgcctgtacaagggctgcgcggagctcggccttgcgccgcggtccaagagcgtcttcaccgagacgcccgcggactggttctcggagatcttcggcaagctcgtggcctcgcccaagacgtggacgagcaactacgacgtcttccacatccgcaacgaggctccgctcgacctccaggacatccctgcgcccccgggtcttaagcagcccgagatggtggcgtacgacgactgggtcgatgccttccaaaaggaggcggccaagcccaactcgaccaacccgctggcgcctctgcgcggcttcgtggccaagggcgaatttgcgcagctgccccagtttgaccagacgcacacgaaggaggtgctgggcgacctgtggacggagccgcccgcgtaccgcttcgagctcctctaagcaaccatccctctgggctaaacgatgtggttctgtgctatggatctctgatggcgtttcgttcccctctacgtttcccctctctttcctggttttccccgctacttgacttacgggtgggg

SEQ ID NO: 2 - DNA, 6042 bp, synthetic construct
  source 1..6042: mol_type = other DNA
atcgcatgcaggtagataacttttacgaacaaagggttacgccctggcttccttgcattggcccaggccccgtgatagccgacgctgacccaagcaatgcggcgcattgacatctggtgggcgcaaggaagggtagcattgtaaataatcgtaggggctgtgacacgcaagacgattcgattcgcgcgggcgttgggttgtccgaatcgcgagaagttgggtgtgtctttccgggcagtctggtctattctcccaactactaccatcagctttggtatcctcgttgttgttgtctgacagaaacgccacttttgtctgacgcgccgttcatattctccgctggttcggttgtatactcattctatcaatcccttcgcgttgtgtttttgaagtgagcgccacacttctttcattcaacgcttgcacctgcatcttccttttccatttcccgcaatatgtctagcactacgcccaaccagcaacagcagtcgcaaggccaggacgaacgcccgcctagcatctcggtggtcggtatcggcctccgtcttcctggagcacgcgacaagaaggaatactttgagctgctcaaggagggccgctgtgccattggccgtgttccggccgaccgttggaacgctgaggcgatgactcaggacggtggtgttcctggcaaggtgatcacggaccagggtggtttcgttgacccccacacggacgttgatacgctcgagtttggtatctcgcccgccgaggcgcgccagatcgacccgcaccaagtcgtgctggttgagtgcgtgtaccaggcactcgaggactcgggtatcaactaccgtggtaccaacaccggtgtgtacgtcacgggttcgcccgacgtgcacaacctgggtaaggacatgtacgacatgggtccctacagtgcgaccggtgcggccttctcgatgcaggccaaccgtctgtcgtacctgttcgacctgcgtggtccgtcgatctacctcgacacggcttgctcgtcgagtatcacgtgtctgcaccttgcgcgcacggccatcctgcgcggcgactgtgacatggcggtcgtggcggcggtcaacctgattcttgcgccggatgcctcgctgtcgttctcgacgctgggcaccctgtccaagacgggtctctgccacacgttcgacgcgtcggccgacggttacgtccgtggtgagggctgcacggtcattgtgctgcagcgcaccgaggacgccgtgcgcctgggcagccacgtgtactcggagatcactggtacgcacatcaatgcaaacggcaagggcaagagtatcactctgcccgacggcccgcagcagaaggccgctacgatcgccgcctacaaggaggcgaagcgcgacttcagcgagacggtgtacgtcgagtgccacggtacgggtacgccggtcggtgacccgatcgaggcgaacgccgtcggtcaggtcttctcgcctggccgctcgagcgacgacaagctgcgcattggttcgtccaagaccaatgtcggtcacttggagcctgctgcgggtcttgtgggtctgatcaagaactgctacaccctcgacacgggctacctgctgccgcacctgcacttccagacgccgagccccaagatccactgggacgacttccagattcaggtgcagaccgcctatgagccgctccccgagaagaagctgagcaaggacggcaagtttatcgtgtcgctctcctcgttcggtttcggtggtgccaactcgcacacggtcatggagcgtgtgccgaacaaggcgcgcattgagaacttcaaggcgcccgcggcgacggacccgatgcttgtcgcggtgggtgcgctgtcgaaccgtgcggtgacctcgctcgagaactcgatggaggagttctggggcaagtcgccgaacccgatgcactcggagctcatggcccgtaccattacggagcgtgcgctccacggtgcagatcctcggtgcggagaaggcattgttccgcgcgctcaagacgaagggcaacacgccaaagtacggcctgatctatcacgcgtcggccatatcgcgcgccgcgcccaagaacaagggccgcatgtcgcgcttccttgccaacaagatcagcattgcgtgccgcattgactgcttctccgacgcgccctccaccaagtttggcgaggtcctgcacatgcaggtcgaggagcgcctcgccttctacgagacgggcaagcctacgacgaagaatagcgatgccatgcgcaaggcgattgccgctatggaggaggccgccggcgacctcatggacgtcgacgctgacgaggatgctgacgaggacgacgagcctgctgagcccgccaagcccaaggaaaagagcaagagcgccaagaaagaaaagaaggacaagaaggaaaagaaggacaagtccaagtccaaatccaaggcggacgatgtggaaaaggccgcgaaaaaggcggccaaaaaggcggccaaagaggccaaagaggccaagaaggacaagaaaaagaaggatgcctgattcagacctcgatccccgtagtatgccgcgcctagaccggccctcttgtataagtaatagcgatgttgggatacgacggcggcagcgcctggggctcctcgcacacgcctcgcgtgcctagcgtggcttgcatgtgatcggcgctgcctgcgtgccagcgcagcagtctcggccgagaccacgagcctgaggccctgtggcggcatttggagcgtggcctagtaggtccccgtggctgtggcatgagcgtccgacagggggacgggagaggcccgccggagtccctgcaccgactttgattggccttggctgacaaagcccgagaggcagcgattgccagcgatcccgattgtgcgacaccgactcgtgttgacgacgcttgtaacaccaccatcaccatggcggccgccactcttgacgatacggcttaccgttacagaaccagtgtcccaggtgatgctgaagccatcgaagcacttgatggtagtttcacaaccgatactgtcttccgagtcacagccaccggtgatggattcactttgcgagaagtccctgttgatccacctcttactaaagtatttcctgatgatgaatcagatgatgaatcagatgcaggtgaagacggcgatccagactccagaacattcgtcgcttacggtgacgatggagaccttgcaggattcgttgtcgtctcttactccggctggaacagacgtcttaccgtcgaagatatcgaagtcgccccagaacaccgtggtcacggtgtcggtagagctttgatgggtcttgctacagaatttgcacgtgaaagaggtgcaggtcacctctggttggaagtcactaacgttaacgcacctgctatccatgcgtaccgacgtatgggtttcaccctttgtggactggataccgcactgtacgacggaaccgcatctgacggagagcaagctctttatatgagtatgccttgtccataagttttctaacgatgcggcatggaatacttagctcttccatagtggccatactacgatcagtgcccacacggatcgaggaccgatgtaagtcctattcgcgcatgccaaacagcttgccgccgcgatggcggacattggcgaacccggcccgggcatccacacagtcgatcgatctgcattagtcgctgccaacgcacgtttcgcgcatccgggccgaccaggcgataggacgaagccgggggaccgttagcgcacaccccagcacgagcactgcaaaggccaccagggtttccagtctcatctgacggtaagacaggcccacctacatccaagggaatcgtgttttcgtactgcgtcgagcctgtgccaagatctaggctctttccctgcgataggactggtgtcagggaactttacctacactcgtagatcgagtacgcagcatggaaaaacaggactgggtcagacataggagaggacggatggactggaaggtcatgcacgagatctcgcctgcgaacctctggtccatttcgcacctggtcgagcactacgacgatgccgacaacgacggtgcggacggcgaggcggaggctgaccccgtcggcacgctcctgggcaagctgctcaacgtcgacatcgagacggtggacgacaccatgttcctgtccacgctcggtctcgactcgctctcggcctcgaagctgagcgcgatcctcatgagcgagtttggcgcgaccgtcacgcagctgcagctgctcggccccgtgtcgattgctgcgctgcgccagattgtcgccgacgccatcgccgagaaggagggtggcgttcccgagaagggcaacacgggcgatgcgcccgccaagaagacggccgccgcccctggcggtgcgtcggcctcgggtgcggcgccgaccagcaaggtcactgcgccgctcgccgagttcgactacgcctcggaggtcgacaagctcgacgacctgcccaagacggtcgcgcacctgcagccgtttgacccggcgatcctcgagccgagcaagaagcaggaggtgctggtgaccggcgccacgggcttcctgggcagctcggcgctcgaggcggtgctcgaggcgctccccaacgcgcacgtcaccgccctggtccgcggctcgcctgccgagggcctgcagcgcatcaagcagatggccgagaagcgcgagctcaagtcgctcaagtcgatggaccgtgtgtcggtcgtgtcgggcgacgtgagccagccgcacctcggcctgtccgaggaggactggacgaagctcgccgagacggttgacctgatcgtgcacgcgagcggcaaggccgaccacctgatcggctaccagggcatcgcccccgtcaacacctactcgacgcgcgaggtgctgcgcctggcgtcggacaagaaggtcaaggcggtcgccaacattggctcgaccaacatgtggctcagcttcaccgcggagcccaagacggacgaggtggtgatggaggacctcgacctggacacgctcaagacgggcctgttcaacggctactcgcagtccaagtgggtgtcggagatgctcacgcgccgcatgcacgagcgcggtgtgcccgccatgacggtgcgccccggtgtgctcggcggtagcgcctcgggccgctaccaccccaacgaggacagcttcctgtggcgcctgtacaagggctgcgcggagctcggccttgcgccgcggtccaagagcgtcttcaccgagacgcccgcggactggttctcggagatcttcggcaagctcgtggcctcgcccaagacgtggacgagcaactacgacgtcttccacatccgcaacgaggctccgctcgacctccaggacatccctgcgcccccgggtcttaagcagcccgagatggtggcgtacgacgactgggtcgatgccttccaaaaggaggcggccaagcccaactcgaccaacccgctggcgcctctgcgcggcttcgtggccaagggcgaatttgcgcagctgccccagtttgaccagacgcacacgaaggaggtgctgggcgacctgtggacggagccgcccgcgtaccgcttcgagctctaagcaaccatccctctgggctaaacgatgtggttctgtgctatggatctctgatggcgtttcgttcccctctacgtttcccctctctttcctggttttccccgctacttgacttacgggtggggccgcgcagcaacgggttggcgccctagcttgctgccgtcattcctcactctcaccttttcccctctggccatgcatggccctttttgtgacatttgcatggagcaagtttttctctcgtagatagacgtcccttgtttgtggcgagcaatgtacgtcgtttcttcctccataatcaatcacgtgctcctttttccggcccgtggctcgtcgcgtgcgcgggcgtcacagcgcatccgtggtgcgttcgtttgggcgagtgcgcgacgcgggcggtgggaatgtttggcgcgtcgtcctttagtgggttc

SEQ ID NO: 3 - DNA, 20 bp, synthetic construct
  source 1..20: mol_type = other DNA
atactttgagctgctcaagg